(19) United States
(12) Patent Application Publication
(10) Pub. No.: US 2026/0123924 A1
(43) Pub. Date: May 7, 2026

(54) SYSTEMS AND METHODS FOR ROBOTIC ENDOLUMINAL SUTURING INSTRUMENT AND NEEDLE DRIVER

(71) Applicant: Noah Medical Corporation, San Carlos, CA (US)

(72) Inventors: Neil MARKWARDT, Redwood City, CA (US); Daniel NASR-CHURCH, San Diego, CA (US); Benjamin SELLE, Fremont, CA (US)

(21) Appl. No.: 19/426,486

(22) Filed: Dec. 19, 2025

Related U.S. Application Data
(63) Continuation of application No. PCT/US2024/034724, filed on Jun. 20, 2024.
(60) Provisional application No. 63/509,945, filed on Jun. 23, 2023.

(51) Int. Cl. A61B 17/04; A61B 17/06; A61B 34/30

(52) U.S. Cl. CPC A61B 17/0469; A61B 34/30; A61B 2017/0609; A61B 2034/301

(57) ABSTRACT

A suturing instrument is provided. The suturing instrument comprises: a flexible shaft comprising an articulatable bending section; a needle driver located at a distal end of the bending section. The needle driver comprises a first jaw driven by a pull wire to have a translatable movement relative to a second jaw thereby rolling a needle grasped by the first jaw and the second jaw.

### REFERENCE

This application is a continuation of International Application No. PCT/US 2024/034724, filed Jun. 20, 2024, which claims priority to U.S. Provisional Patent Application No. 63/509,945 , filed on Jun. 23, 2023, which is entirely incorporated herein by reference.

### BACKGROUND

Endoscopy procedures use an endoscope to examine the interior of a hollow organ or cavity of the body. Unlike many other medical imaging techniques, endoscopes are inserted into the organ directly via the mouth or other naturally occurring orifices. Flexible endoscopes that can deliver instinctive steering and control are useful in diagnosing and treating diseases that are accessible through any natural orifice in the body. Depending on the clinical indication, the endoscope may be designated as colonoscope, gastroscope, bronchoscope, ureteroscope, ENT scope, and various others. For example, a flexible colonoscope may be intubated to transverse colon for diagnosis and/or surgical treatment.

Endoscopes are traditionally made to be re-usable, which may require thorough cleaning, dis-infection, and/or sterilization after each procedure. In most cases, cleaning, dis-infection, and sterilization may be aggressive processes to kill germs and/or bacteria. Such procedures may also be harsh on the endoscopes themselves. Therefore, the designs of such re-usable endoscopes can often be complicated, especially to ensure that the endoscopes can survive such harsh cleaning, dis-infection, and sterilization protocols. Periodical maintenance and repairs for such re-usable endoscopes may often be needed.

Low cost, disposable medical devices designated for a single-use have become popular for instruments that are difficult to clean properly. Single-use, disposable devices may be packaged in sterile wrappers to avoid the risk of pathogenic cross-contamination of diseases such as HIV, hepatitis, and other pathogens. Hospitals generally welcome the convenience of single-use disposable products because they no longer have to be concerned with product age, overuse, breakage, malfunction, and sterilization. Traditional endoscopes often include a handle that operators use to maneuver the endoscope. For single-use endoscopes, the handle usually encloses the camera, expensive electronics, and mechanical structures at proximal end in order to transmit the video and allow the users to maneuver the endoscope via a user interface. This may lead to high cost of the handle for a single-use endoscope.

An endoscope device may have a working channel allowing tools such as graspers, cutters or suturing instruments to pass through. In another example, a suturing device can be coupled to the distal end of an endoscope, which enables suturing in the gastroesophageal tract of a patient. However, current suturing devices are designed for manual endoscope devices such as laparoscopy. In the past, suturing of bodily organs or tissue through endoscopic surgery was achieved through the use of a sharp metal suture needle which had attached at one of its ends a length of suture material. The needle is delivered to the target location via a needle delivery device, then grasped by a needle driver attached to an end of another instrument for suturing operation. In a non-wristed instrument, getting a suture needle oriented properly in the needle driver jaws presents significant challenges, adding to procedure time and cognitive load for the user. Grasping the needle at the proper location may not be hard, but having the needle in a desired angular orientation can be challenging. Current solution for adjusting the orientation of the needle may be achieved via a wrist at the distal end of the instrument resulting in extra cost and increased dimension of the instrument.

### SUMMARY

Recognized herein is a need for suturing devices suitable for robotic endoscopic platforms or used endoluminally with reduced size and improved needle orientation control.

In an aspect of the present disclosure, a suturing instrument is provided. The suturing instrument comprises a flexible shaft comprising an articulatable bending section and a needle driver located at a distal end of the bending section. The needle driver comprises a first jaw driven to have a translatable movement relative to a second jaw thereby rolling a needle grasped by the first jaw and the second jaw into a desired orientation.

In some embodiments, the first draw is driven to translate relative to the second draw by a first pull wire that is extended through the flexible shaft. In some cases, a proximal end of the first pull wire is coupled to a handle of the suturing instrument. In some instances, the handle is releasably coupled to a robotic support via a first instrument driving mechanism. In some cases, the first draw or the second draw is driven to open or close by a second pull wire that is extended through the flexible shaft.

In some embodiments, the suturing instrument is inserted through a working channel of a flexible robotic endoscope. In some cases, the flexible robotic endoscope comprises an articulable bending section. In some instances, the flexible robotic endoscope is releasably coupled to a robotic support via a second instrument driving mechanism. For example, the articulable bending section is actuated by the second instrument driving mechanism.

In some embodiments, the needle is rolled into the desired orientation by an angle. For instance, a range of the angle is determined based on a translational travel distance between the first draw and the second draw.

In another aspect, a robotic endoluminal suturing instrument is provided. The robotic endoluminal suturing instrument comprises: a flexible shaft comprising an articulatable bending section, where an articulating motion of the articulatable bending section is driven by a first pull wire; a needle driver located at a distal end of the bending section, wherein the needle driver comprises a first jaw driven by a second pull wire to have a translatable movement relative to a second jaw thereby rolling a needle grasped by the first jaw and the second jaw into a desired orientation; and a handle configured to releasably couple the robotic endoluminal suturing instrument to an instrument driving mechanism, wherein the instrument driving mechanism actuates the first pull wire and the second pull wire.

In some embodiments, the handle is releasably coupled to a robotic support via a first instrument driving mechanism. In some embodiments, the first draw or the second draw is driven to open or close by a third pull wire that is extended through the flexible shaft.

In some embodiments, the robotic endoluminal suturing instrument is inserted through a working channel of a flexible robotic endoscope. In some cases, the flexible robotic endoscope comprises an articulable bending section. In some instances, the flexible robotic endoscope is releasably coupled to a robotic support via a second instrument driving mechanism. For example, the articulable bending section is actuated by the second instrument driving mechanism.

In some embodiments, the needle is rolled into the desired orientation by an angle. In some cases, a range of the angle is determined based on a translational travel distance between the first draw and the second draw.

It should be noted that the provided modular endoscope components and various components of the device can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others. The devices and systems can be used in any subject that may or may not involve human body, animal, or tissue.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically shows an example of a needle driver, in accordance with some embodiments of the present disclosure.

FIG. 2 schematically shows an example of a suturing instrument, in accordance with some embodiments of the present disclosure.

FIGS. 3-9 shows an example of a needle driver, in accordance with some embodiments of the present disclosure.

FIG. 10 illustrates an example of a flexible endoscope.

FIG. 11 shows an example of a robotic endoscope (e.g., gastroscope or colonoscope).

FIG. 12A and FIG. 12B show an example of an instrument driving mechanism (IDM) providing mechanical interface to the handle portion of the robotic endoscope.

FIG. 13 shows an example of a distal tip of an endoscope.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In an aspect of the present disclosure, a suturing instrument is provided. The suturing instrument comprises: a flexible shaft comprising an articulatable bending section; a needle driver located at a distal end of the bending section. The needle driver comprises a first jaw driven by a pull wire to have a translatable movement relative to a second jaw thereby rolling a needle grasped by the first jaw and the second jaw.

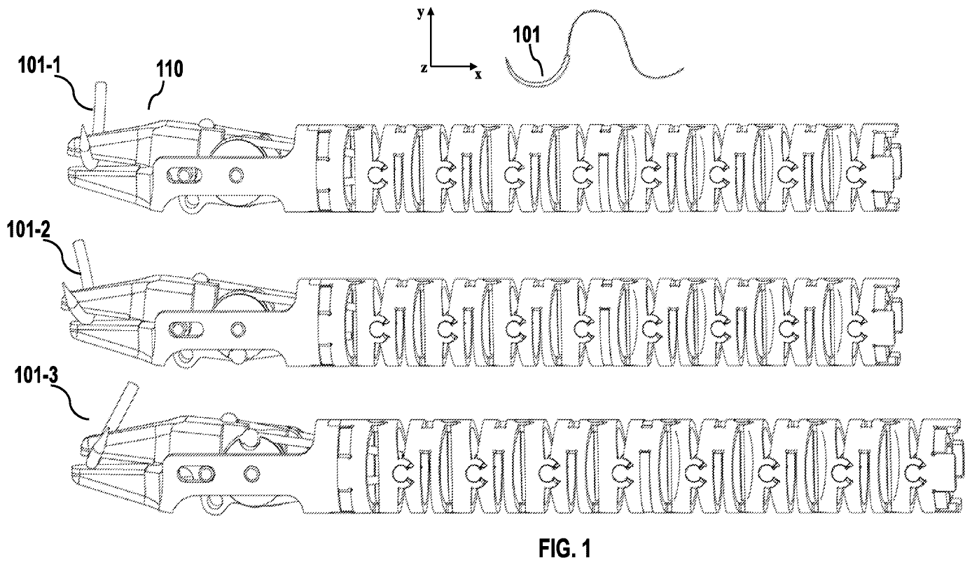

As described above, in a non-wristed instrument (without the capability to control orientation of a needle via motion of a wrist at distal end), getting a suture needle oriented properly in a needle driver has significant challenges, adding to procedure time and cognitive load for the user. User may be capable of grasping the needle at the proper location by a needle driver jaw, but grasping the needle in a desired angular orientation can be difficult. As illustrated in FIG. 1, a suturing needle 101 may be driven within a needle plane (e.g., XY plane). The needle may be a curved needle defining a needle plane. For example, the needle may be a long shouldered needle, a standard ½ circle, or 180° needle that an orientation of the needle plane can be critical. Those skilled in the art will understand that various needle wire diameters, needle bend radii, needle cross sections, and suture materials are all adaptable to be used in the devices described herein. The orientation of the needle (e.g., orientation of the XY plane) may affect driving the curved needle in a circular arc path. As described above, in order to load the needle in the needle driver at the desired angular orientation, a user may have to first oriented the needle properly then grasp the needle by the needle driver jaws at the orientation. This process can be challenging, adding to procedure time and cognitive load for the user. Other solution may involve including a wrist at the distal end of the instrument so an orientation of the needle can be adjusted via the rotational motion of the wrist. However, such device results in extra cost and increased dimension.

The present disclosure addresses the above issue by providing an improved needle driver. The needle driver may be located at a distal end of a bending section of an instrument that can grasp a needle and adjust an orientation of the needle after grasping. The needle driver may comprise a first jaw driven by a pull wire to have a translatable movement relative to a second jaw thereby rolling a needle grasped by the first jaw and the second jaw. As shown in FIG. 1, the needle driver 110 may comprise a pair of jaws that are capable of grasping a needle and rolling the needle into any desired orientation. Unlike traditional needle driver having a pair of juxtaposed jaws pivotally associated with one another, the needle driver 110 herein may add a translation function to one of the jaws, which can roll the needle into the desired orientation after grasping. As illustrated in FIG. 1, the translating jaw may have a predefined translational travel distance to rotate a needle in either direction from the starting orientation (e.g., rotated from an initial orientation 101-1 to any degree in a first direction 101-2 or a second direction 101-3). The range of orientation can be any number of angular degrees depending on the desired application. For example, the orientation of the needle may be adjusted within any angular range from 20 degree to 180 degree.

The needle driver 110 may be an end effector attached to any articulable instrument. The simplified design may allow the needle driver to be attached to a distal end of an instrument with a small diameter (e.g., 7 mm, 7 mm, 6 mm, 5 mm, 4.5 mm, 4 mm or smaller instrument diameter). It should be noted that the needle driver may have a dimension (e.g., diameter) beneficially allowing the needle driver to be integrated into any instrument as an end effector. As described above, the needle driver may comprise a translating jaw, which is controlled by a degree of freedom (DOF) which is independent of the jaw open/close DOF. This beneficially allows the suturing instrument to grasp a needle in an arbitrary orientation and then use the translation DOF of the jaw to roll the needle into a desired orientation for suturing.

The embodiments disclosed herein can be combined in one or more of many ways to provide improved diagnosis and therapy to a patient. The disclosed embodiments can be combined with existing methods and apparatus to provide improved treatment, such as combination with methods of pulmonary diagnosis, surgery and surgery of tissues and organs, manual instrument or robotic instrument, for example. It is to be understood that any one or more of the structures and steps as described herein can be combined with any one or more additional structures and steps of the methods and apparatus as described herein, the drawings and supporting text provide descriptions in accordance with embodiments.

In some cases, a needle may be delivered using a delivery mechanism to a target location to be grabbed or grasped by the needle instrument. In some cases, a needle may be delivered using a needle delivery device that is an instrument separately and independently controlled from the suturing instrument. For instance, a needle and suture may be loaded into a specialized holder which is passed through an instrument channel of the endoscope (e.g., colonoscope) that is separate from an instrument channel the suturing instrument is passed through. The needle delivery device may deliver the needle to a location opposite the needle driver at a target site. A release is actuated to allow the needle to be grasped by the needle driver and removed from the needle delivery device. In some cases, the needle delivery device may be articulatable such as comprising an articulatable bending section to allow it to be positioned for ease of grasping the needle. The articulation and release may be actuated robotically or manually. In some cases, the needle delivery device may be passed through the auxiliary channel in the colonoscope. Alternatively, instead of using a separate needle delivery device, the needle may be preloaded to the suturing instrument such as by clamped to the needle driver jaws before it is introduced into the working channel for the suturing instrument, then the orientation of the needle may be further adjusted once the suturing instrument reaches its target site or destination.

While exemplary embodiments will be primarily directed at a suturing device or system for colonoscope or gastroscope, one of skill in the art will appreciate that this is not intended to be limiting, and the devices described herein may be used for other therapeutic or diagnostic procedures and for being placed in various anatomical regions of a patient's body, through a passage way, or in any subject. The provided suturing device or system can be utilized in urology, gynecology, rhinology, otology, laryngoscopy, gastroenterology with the endoscopes, combined devices including endoscope and instruments, endoscopes with localization functions, one of skill in the art will appreciate that this is not intended to be limiting, and the devices described herein may be used for other therapeutic or diagnostic procedures and in other anatomical regions of a patient's body, such as such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat, and various others, in the forms of: BronchoScope, NeuroendoScope, EncephaloScope, OphthalmoScope, OtoScope, RhinoScope, LaryngoScope, GastroScope, EsophagoScope, BronchoScope, ThoracoScope, PleuroScope, AngioScope, MediastinoScope, NephroScope, GastroScope, DuodenoScope, CholeodoScope, CholangioScope, LaparoScope, AmioScope, UreteroScope, HysteroScope, CystoScope, ProctoScope, ColonoScope, ArthroScope, SialendoScope, Orthopedic Endoscopes, and others, in combination with various tools or instruments. The devices and systems can be used in any subject that may or may not involve human body, animal, or tissue.

The systems and apparatuses herein can be combined in one or more of many ways to provide improved diagnosis and therapy to a patient. Systems and apparatuses provided herein can be combined with existing methods and apparatus to provide improved diagnosis, surgery operations of various tissues and organs, for example. It is to be understood that any one or more of the structures and steps as described herein can be combined with any one or more additional structures and steps of the methods and apparatus as described herein, the drawings and supporting text provide descriptions in accordance with embodiments.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the terms distal and proximal may generally refer to locations referenced from the apparatus, and can be opposite of anatomical references. For example, a distal location of a primary shaft or catheter may correspond to a proximal location of an elongate member of the patient, and a proximal location of the primary sheath or catheter may correspond to a distal location of the elongate member of the patient.

### Suturing Instrument and Needle Driver

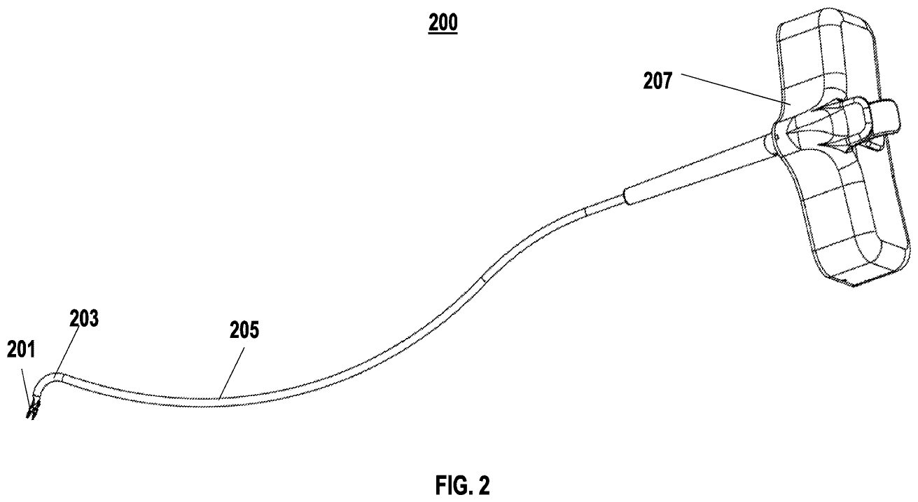

FIG. 2 schematically shows an example of a suturing instrument 200, in accordance with some embodiments of the present disclosure. The suturing instrument 200 may comprise an end effector 201 located at a distal end of an elongate member. The end effector may comprise a needle driver as described in FIG. 1 and elsewhere herein. The elongate member may be an articulatable, flexible member comprising a bending section 203, a flexible shaft 205 and a proximal end 207. The proximal end 207 may be a handle that is releasably attached to a robotic support. In some cases, the proximal end 207 may comprise driving components (e.g., pulley) that are releasably coupled to an instrument driving mechanism to drive an operation of the end effector (e.g., open/close of jaws to grasp needle, translational motion of a jaw to adjust orientation of the grasped needle, etc.) and/or the motion (e.g., articulation) of the bending section 203.

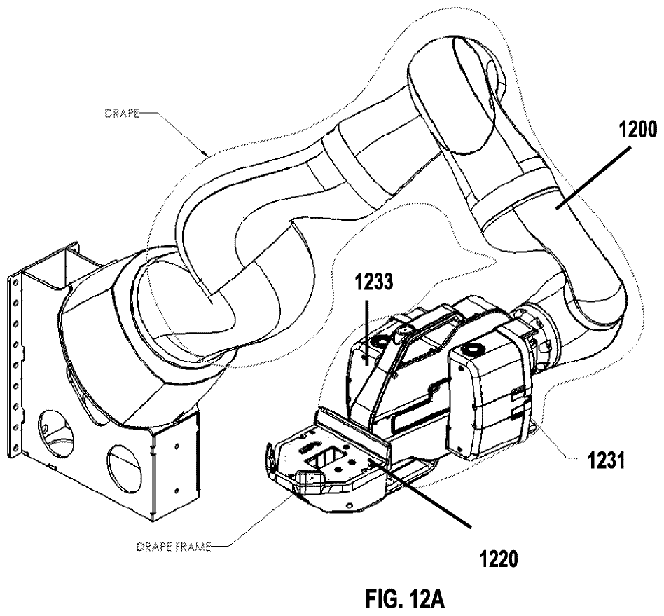

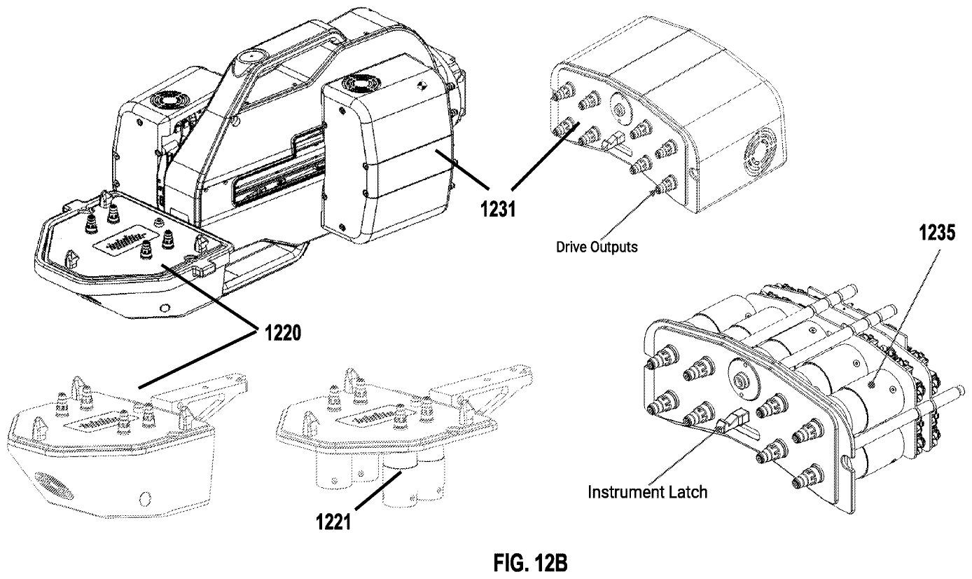

In some cases, the proximal end 207 may comprise a mechanical interface to allow the suturing instrument to be releasably coupled to an instrument driving mechanism attached to a robotic support or a hand-held controller. The instrument driving mechanism (IDM) can be the same as the IDM 1231, 1233 as illustrated in FIG. 12A and FIG. 12B. The IDM may comprise a set of motors 1235 that are actuated to rotationally drive a set of pull wires of the elongate member. The proximal end 207 may be mounted onto the instrument drive mechanism 1231 or 1233 so that its pulley/capstans assemblies are driven by the set of motors. The number of pulleys may vary based on the pull wire configurations. In some cases, at least one, two, three, four, or more pull wires may be utilized for articulating the flexible suturing instrument and for driving the motion of the needle end effector. In some cases, one pull wire may be coupled to and driven by a pulley. For example, one pull wire may drive to open the jaw of the end effector, one pull wire may drive to close the jaw, one pull wire may drive to translate the jaw forward and one pull wire may drive the jaw to translate rearward. In some cases, more than one wires may be coupled to a driven pulley. For example, two or more wires may be coupled to the same driven pulley antagonistically to drive the needle driver end effector motion such that rotation of the pulley provides tension to one wire(s) while slacking the other(s).

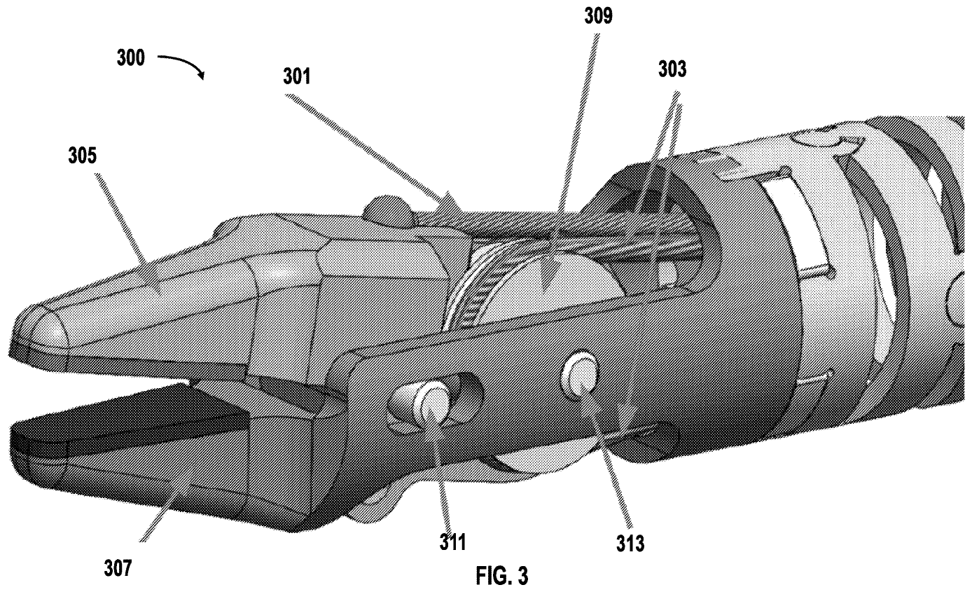

The bending section 203 may be articulated in two or more degrees of freedom. The articulation of the bending section 203 may be controlled by applying force to the distal tip portion via the one or multiple pull wires. A distal end of the one or more pull wires may be attached to the distal end of the suturing instrument 200. In the case of multiple pull wires, pulling one wire at a time may change the orientation of the end effector 201 to pitch up, down, left, right or any direction needed. In some cases, the pull wires may be anchored at the distal tip portion of the suturing instrument 200, running through the bending section, and entering the proximal end they are coupled to a driving component (e.g., pulley). This pulley may interact with an output shaft from the robotic system (such as shown in FIG. 12B). In some cases, one or more of the pull wires may be utilized as the needle drive mechanism (e.g., jaw open cable 301, jaw translation cable 303 in FIG. 3) to drive forward and backward motion of a jaw relative to another jaw and open/close motion of the jaw in the end effector.

In some embodiments, the proximal end or portion of one or more pull wires may be operatively coupled to various mechanisms (e.g., gears, pulleys, capstans, etc.) in the proximal end. The pull wire may be a metallic wire, cable or thread, or it may be a polymeric wire, cable or thread. The pull wire can also be made of natural or organic materials or fibers. The pull wire can be any type of suitable wire, cable or thread capable of supporting various kinds of loads without deformation, significant deformation, or breakage. The distal end/portion of one or more pull wires may be anchored or integrated to the distal portion of the suturing instrument 200, such that operation of the pull wires by the control unit may apply force or tension to the distal portion which may steer or articulate (e.g., up, down, pitch, yaw, or any direction in-between) at least the distal portion (e.g., flexible section) of the suturing instrument. The pull wires may be made of any suitable material such as stainless steel (e.g., SS316), metals, alloys, polymers, nylons or biocompatible material. In some embodiments, different pull wires may be made of different materials for varying the load bearing capabilities of the pull wires.

The end effector 201 may comprise a needle driver device. The needle driver device may be attached to the distal portion of the suturing instrument 200. The needle driver device may have two (e.g., roll and translation), three (e.g., roll and articulation), four (e.g., roll, articulation and translation) or more degrees of freedom via a manipulation of the suturing instrument. For example, the needle driver device may have a roll movement (e.g., rotatable about the longitudinal axis of the elongate member), articulatable about two axes (e.g., via the articulation of the bending section) and may have translational movement (e.g., insertion and retraction of the device). In some cases, the needle device may be integrated to the distal portion of the bending section 203 and may not have roll movement relative to the bending section. In some cases, a roll movement of the needle device may be achieved via the roll movement of the elongate member, a wrist located at the distal end of the bending section 203 or a combination of both. The needle driver end effector 201 as described herein may beneficially allow for adjusting orientation of a needle with or without a wrist at the distal end of the instrument.

The present disclosure provides an improved needle driver device or needle driver end effector with improved needle orientation control that can be used with an endoscope or colonoscope to perform operations related to suturing during upper and lower gastrointestinal (GI) tract endoscopy, gastric endoscopy, small bowel endoscopy or other procedures. The reduced size of the end effector may beneficially ease insertion, manipulation, and retraction of an endoscope during colonoscopy. In particular, the devices and methods of the present disclosure provides an improved needle driver device with needle orientation control allowing for a precise needle loading/operation control for robotic endoluminal surgical platforms.

As mentioned above, traditional needle driver may have a pair of juxtaposed jaws pivotally associated with one another to grasp a needle. Once the needle is grasped by the pair of jaws, the orientation of the needle is not adjustable. However, the needle driver herein may allow for adjustment of needle angular orientation after grasping by adding a translation function to at least one of the jaws. The translational movement between the pair of jaws may roll the needle into a desired orientation after grasping. In some embodiments of the needle driver device, at least one of the pair of jaws may be driven to translate forward/backward relative to another jaw thereby rolling a needle into a desired orientation.

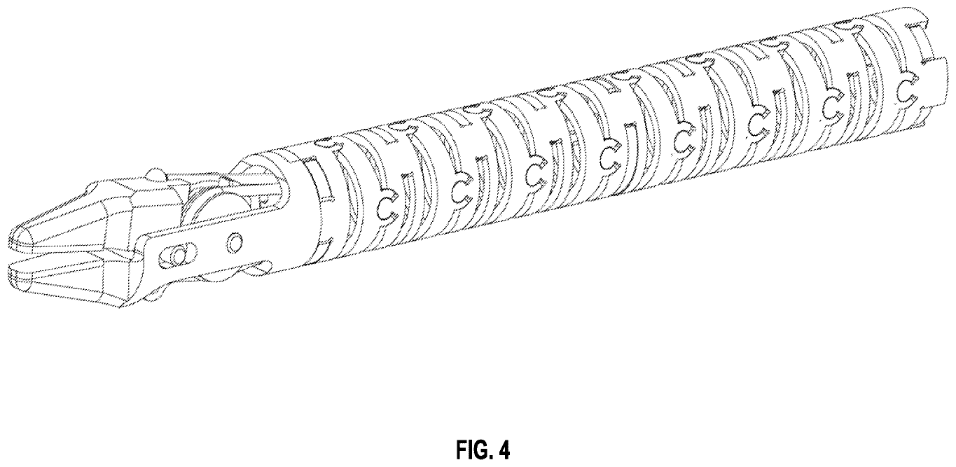

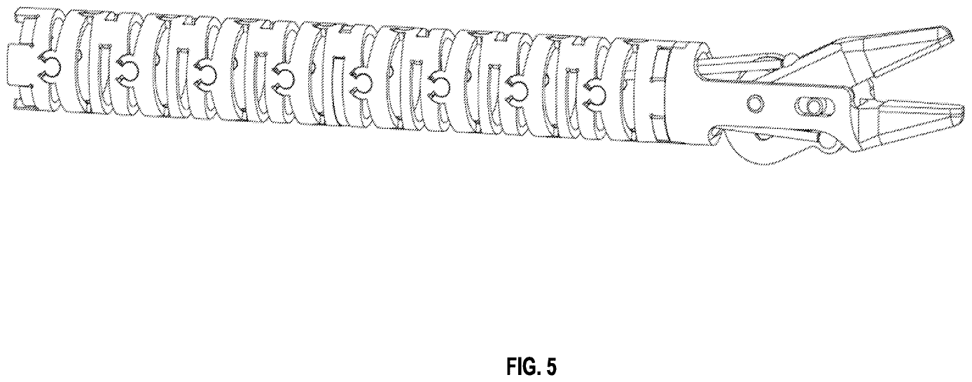

FIGS. 3-6 shows an example of a needle driver 300 in accordance with some embodiments of the present disclosure. In the illustrated example, the needle driver may comprise a pair of jaws 305, 307. One of the pair of jaws may be translating jaw and the other one may be a fixed jaw. For example, the jaw 305 may be driven to translate forward/backward relative to the other jaw 307. Such translational movement between the pair of jaws may effectuate a roll motion of a needle (e.g., needle 101 in FIG. 1) grasped by the jaws.

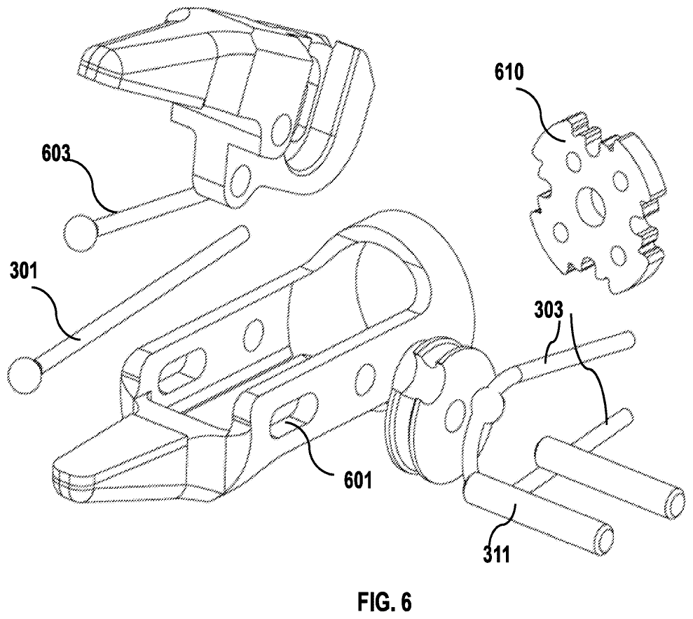

The translatable or movable jaw 305 may be driven to translate along the tool axis direction relative to the fixed jaw 307. It should be noted that either jaw can be the translatable jaw or the fixed jaw. A roll angle range for the needle (once grasped by the jaws) may be dependent on the translational travel range of the jaws and a diameter of the needle. For example, as shown in the example, a jaw hinge pin 311 may be slidable within a slot. In the exploded view of FIG. 6, the length of the slot 601 may determine the translational travel distance of the jaws or the rotation angle range for the needle. A range of the angle is determined based on a translational travel distance between the first draw and the second draw. As an example, the translatable jaw may be permitted to travel within the slot to rotate a 0.6 mm diameter needle 90 degrees in either direction from a starting position as an end effector for an instrument diameter (e.g., with about 4.5 mm diameter).

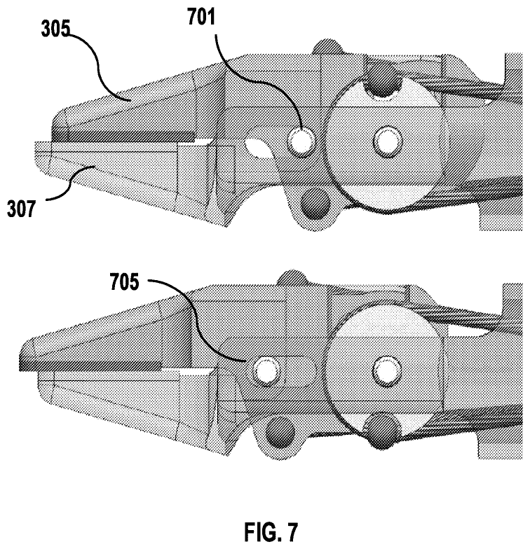

FIG. 7 shows examples of the movable jaw 305 translating to the maximum positions 701, 705 (relative to the fixed jaw 307) that correspond to the two ends of the slot that the jaw hinge pin can reach.

As described above, the actuation or driving mechanism of the needle driver end effector may comprise one or more pull wires. The one or more pull wires may comprise one pull wire to open a jaw, one pull wire to close the jaw, one pull wire to translate the jaw forward and one pull wire to translate the jaw rearward. For instance, a first pull wire 301 may be connected to the movable jaw 305 to control an opening of the movable jaw. Pulling the first pull wire 301 may drive the movable jaw 305 to open. A second pull wire such as the pull wire 603 in FIG. 6 may be attached to the movable jaw 305 to control the closing of the movable jaw. For example, pulling the second pull wire 603 may close the movable jaw and grasp a needle that is placed between the pair of jaws. Alternatively, a single pull wire may be utilized to control both the open and close motion of the jaw.

In some cases, a pair of translation cables or pull wires e.g., pull wires 303, may rotate a cam 309. The pull wires for the translational movement may be connected to the cam 309 via any suitable structure. For example, the pull wire may be crimped to the cam. Alternatively, a single pull wire may be utilized to control the translational movement of the movable jaw.

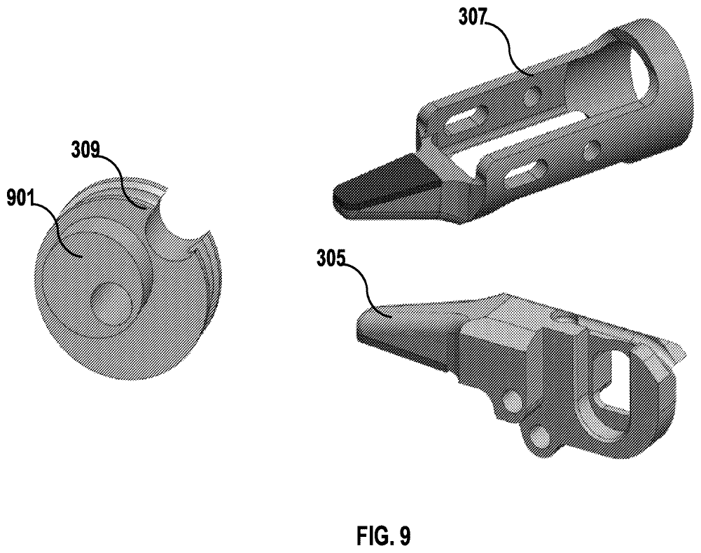

The translation cam 309 may have an eccentric lobe that engages the movable jaw. FIG. 9 shows an example of the translation cam 309. The eccentric lobe 901 may cause the movable jaw 305 to translate along the tool axis direction as the cam is rotated about the cam axel pin 313 and the jaw hinge pin 311 is free to move within the slot 601 as described elsewhere herein.

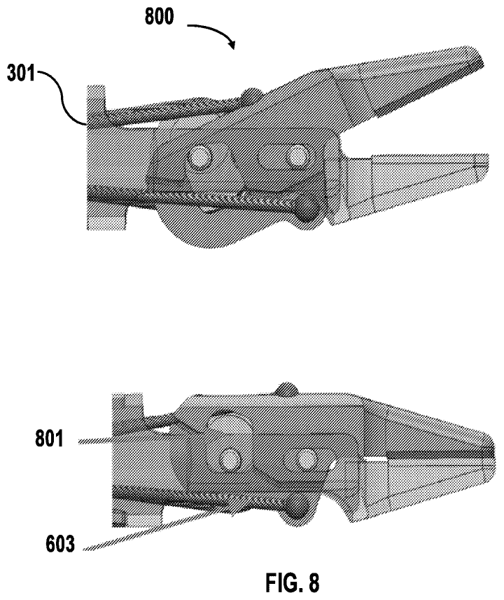

As described above, the needle driver may comprise a translating jaw, which is controlled by a degree of freedom (DOF) that is independent of the jaw open/close DOF. This beneficially allows a user to grasp a needle in an arbitrary orientation and then use the translation DOF of the jaw to roll the needle into a desired orientation for suturing. In some cases, the driving mechanism may be located on opposing sides of the needle driver device. For example, the driving mechanism for controlling open/close of the jaw may be located on a first side such as shown in FIG. 8 where the pull wire 603 for closing the jaw is located on the right side, and the pull wire and cam 801 for controlling translational movement of the jaw is located on the second side or the left side. In some cases, the needle driver device may comprise a component matching a configuration of the driving mechanism or the plurality of pull wires. As shown in FIG. 6, a component 610 may comprise a plurality of holes to hold the plurality of pull wires in place. This component 610 may beneficially capture the plurality of pull wires to constraint a radial motion of the pull wires while allowing them to translate distally/proximally. The driving mechanism can have any suitable configuration. For example, the pull wires for driving the open/close DOF and translation DOF may be placed on opposing sides of the needle driver or on the same sides of the needle driver.

It should be noted that the needle driver device can have various configurations. For instance, instead of one movable jaw that can both open/close and translate relative to a fixed jaw, the needle driver device may comprise a first jaw that is controlled to open/close, and a second jaw that is controlled to translate relative to the first jaw. In another alternative embodiment, both jaws may be controlled to open/close, and one of the pair of jaws may be controlled to translate. In a further alternative embodiment, both of the jaws may be controlled to open/close and translate. For instance, the two jaws may be controlled to translate in opposing directions thereby increasing an overall travel distance.

The translatable jaw design may beneficially allow for adjusting needle orientation without increasing the size of the needle end effector. The reduced dimension (e.g., diameter) of the end effector may allow for an improved anatomical access and may permit use in conjunction with additional tissue manipulation instruments (e.g., graspers) for tissue placement and suture management. Additionally, an endoluminal device with the provided end effector may have an overall reduced diameter allowing for higher quality and more rapid lesion closure than existing technologies when used in conjunction with a robotic surgical platform. For example, the outer diameter of the needle instrument may be no greater than 5 millimeters (mm), 4.9 mm, 4.8 mm, 4.7 mm, 4.6 mm, 4.5 mm, 4.4 mm, 4.3 mm, 4.2 mm, 4.1 mm, 4 mm, 3.5 mm, 3 mm, any number in between the above numbers or any number greater than 5 mm or small than 3 mm. The total length of the needle end effector may be no greater than 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, any number in between the above numbers or any number greater than 30 mm or small than 15 mm.

### Flexible Endoscope

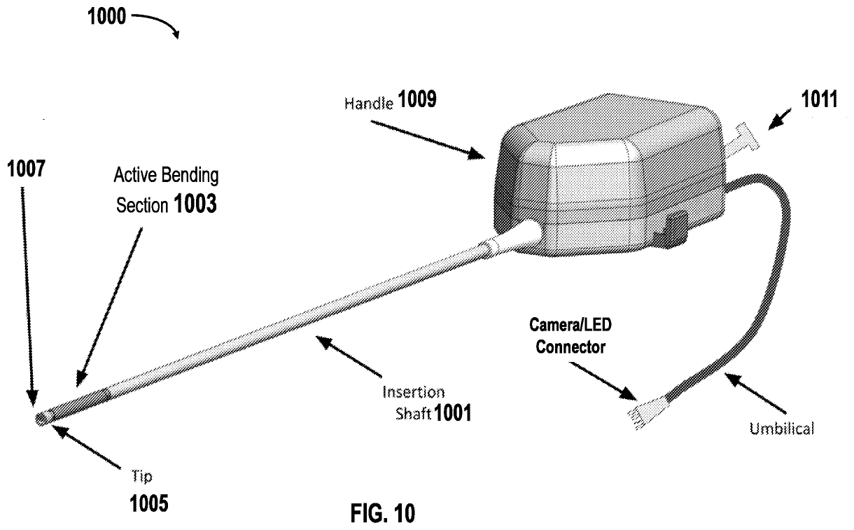

The provided suturing instrument may be utilized by any robotic endoluminal systems or platforms. In an aspect of the present disclosure, the suturing instrument may be inserted through a working channel of a flexible endoscope to perform suturing operations as described above. The suturing instrument may be independently steerable from the endoscope. For example, the suturing instrument shaft may be advanced and retracted and rotated relative to the flexible endoscope. FIG. 10 illustrates an example of a flexible endoscope 1000, in accordance with some embodiments of the present disclosure. As shown in FIG. 10, the flexible endoscope 1000 may comprise a handle/proximal portion 1009 and a flexible elongate member to be inserted inside of a subject. The flexible elongate member can be the same as the one described above. In some embodiments, the flexible elongate member may comprise a proximal shaft (e.g., insertion shaft 1001), steerable tip (e.g., tip 1005), a steerable section (active bending section 1003) and a proximal shaft section 1001. The endoscope 1000 may also be referred to as steerable catheter assembly as described elsewhere herein. In some cases, the endoscope 1000 may be a single-use robotic endoscope. In some cases, the entire catheter assembly may be disposable. In some cases, at least a portion of the catheter assembly may be disposable. In some cases, the entire endoscope may be released from an instrument driving mechanism and can be disposed of In some embodiments, the endoscope may contain varying levels of stiffness along the shaft, as to improve functional operation.

The endoscope or steerable catheter assembly 1000 may comprise a handle portion 1009 that may include one or more components configured to process image data, provide power, or establish communication with other external devices. For instance, the handle portion may include a circuitry and communication elements that enables electrical communication between the steerable catheter assembly 1000 and an instrument driving mechanism (not shown), and any other external system or devices. In another example, the handle portion 1009 may comprise circuitry elements such as power sources for powering the electronics (e.g., camera, electromagnetic sensor and LED lights) of the endoscope.

The one or more components located at the handle may be optimized such that expensive and complicated components may be allocated to the robotic support system, a hand-held controller or an instrument driving mechanism thereby reducing the cost and simplifying the design of the disposable endoscope. The handle portion or proximal portion may provide an electrical and mechanical interface to allow for electrical communication and mechanical communication with the instrument driving mechanism. The instrument driving mechanism may comprise a set of motors that are actuated to rotationally drive a set of pull wires of the catheter. The handle portion of the catheter assembly may be mounted onto the instrument drive mechanism so that its pulley/capstans assemblies are driven by the set of motors. The number of pulleys may vary based on the pull wire configurations. In some cases, one, two, three, four, or more pull wires may be utilized for articulating the flexible endoscope or catheter.

The handle portion may be designed allowing the robotic endoscope to be disposable at reduced cost. For instance, classic manual and robotic endoscopes may have a cable in the proximal end of the endoscope handle. The cable often includes illumination fibers, camera video cable, and other sensors fibers or cables such as electromagnetic (EM) sensors, or shape sensing fibers. Such complex cable can be expensive adding to the cost of the endoscope. The provided robotic endoscope may have an optimized design such that simplified structures and components can be employed while preserving the mechanical and electrical functionalities. In some cases, the handle portion of the robotic endoscope may employ a cable-free design while providing a mechanical/electrical interface to the catheter.

The electrical interface (e.g., printed circuit board) may allow image/video data and/or sensor data to be received by the communication module of the instrument driving mechanism and may be transmitted to other external devices/systems. In some cases, the electrical interface may establish electrical communication without cables or wires. For example, the interface may comprise pins soldered onto an electronics board such as a printed circuit board (PCB). For instance, a receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

In some cases, the handle portion 1009 may comprise one or more mechanical control modules such as a lure 1011 for interfacing the irrigation system/aspiration system. In some cases, the handle portion may include a lever/knob for articulation control. Alternatively, the articulation control may be located at a separate controller attached to the handle portion via the instrument driving mechanism.

The endoscope may be attached to a robotic support system or a hand-held controller via the instrument driving mechanism. The instrument driving mechanism may be provided by any suitable controller device (e.g., hand-held controller) that may or may not include a robotic system. The instrument driving mechanism may provide mechanical and electrical interface to the steerable catheter assembly 1000. The mechanical interface may allow the steerable catheter assembly 1000 to be releasably coupled to the instrument driving mechanism. For instance, the handle portion of the steerable catheter assembly can be attached to the instrument driving mechanism via quick install/release means, such as magnets, spring-loaded levers and the like. In some cases, the steerable catheter assembly may be coupled to or released from the instrument driving mechanism manually without using a tool. Details about the instrument driving mechanism are described later herein.

In the illustrated example, the distal tip of the catheter or endoscope shaft is configured to be articulated/bent in two or more degrees of freedom to provide a desired camera view or control the direction of the endoscope. As illustrated in the example, imaging device (e.g., camera), position sensors (e.g., electromagnetic sensor) 1007 is located at the tip of the catheter or endoscope shaft 1005. For example, line of sight of the camera may be controlled by controlling the articulation of the active bending section 1003. In some instances, the angle of the camera may be adjustable such that the line of sight can be adjusted without or in addition to articulating the distal tip of the catheter or endoscope shaft. For example, the camera may be oriented at an angle (e.g., tilt) with respect to the axial direction of the tip of the endoscope with the aid of an optical component.

The distal tip 1005 may be a rigid component that allows for positioning sensors such as imaging devices (e.g., camera) and other electronic components (e.g., LED light source) being embedded at the distal tip. Depending on the type of the endoscope, the distal tip may comprise other sensors such as electromagnetic (EM) sensors or inertial measurement units.

The robotic endoscope may or may not have real-time EM tracking capability. In the case that the robotic endoscope is embedded with EM sensor, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. For example, the EM field generator may be positioned close to the patient during a procedure to locate the EM sensor position in 3D space or may locate the EM sensor position and orientation in 5D or 6D space. This may provide a visual guide to an operator when driving the endoscope towards the target site.

The endoscope may have a unique design in the elongate member. In some cases, the active bending section 1003 and the proximal shaft 1001 of the endoscope may consist of a single tube that incorporates a series of cuts (e.g., reliefs, slits, etc.) along its length to allow for improved flexibility, a desirable stiffness as well as the anti-prolapse feature (e.g., features to define a minimum bend radius).

As described above, the active bending section 1003 may be controlled to allow for bending in two or more degrees of freedom (e.g., articulation). A greater bending degree such as 180 and 270 degrees (or other articulation parameters for clinical indications) can be achieved by the unique structure of the active bending section. In some cases, the active bending section and/or the passive section may be fabricated separately as a modular component and assembled to the proximal shaft. In some cases, the cut patterns of the active bending and passive sections may be different such that at least the minimum bend radius of the two sections may be different. In some cases, a variable minimum bend radius along the axial axis of the elongate member may be provided such that an active bending section or the passive section may comprise two or more different minimum bend radii.

The articulation of the endoscope may be controlled by applying force to the distal end of the endoscope via one or multiple pull wires. The one or more pull wires may be attached to the distal end of the endoscope. In the case of multiple pull wires, pulling one wire at a time may change the orientation of the distal tip to pitch up, down, left, right or any direction needed. In some cases, the pull wires may be anchored at the distal tip of the endoscope, running through the bending section, and entering the handle where they are coupled to a driving component (e.g., pulley). This handle pulley may interact with an output shaft from the robotic system.

In some embodiments, the proximal end or portion of one or more pull wires may be operatively coupled to various mechanisms (e.g., gears, pulleys, capstans, etc.) in the handle portion of the catheter assembly. The pull wire may be a metallic wire, cable or thread, or it may be a polymeric wire, cable or thread. The pull wire can also be made of natural or organic materials or fibers. The pull wire can be any type of suitable wire, cable or thread capable of supporting various kinds of loads without deformation, significant deformation, or breakage. The distal end/portion of one or more pull wires may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The pull wires may be made of any suitable material such as stainless steel (e.g., SS316), metals, alloys, polymers, nylons or biocompatible material. Pull wires may be a wire, cable or a thread. In some embodiments, different pull wires may be made of different materials for varying the load bearing capabilities of the pull wires. In some embodiments, different sections of the pull wires may be made of different material to vary the stiffness and/or load bearing along the pull. In some embodiments, pull wires may be utilized for the transfer of electrical signals.

The proximal design may improve the reliability of the device without introducing extra cost allowing for a low-cost single-use endoscope. In another aspect of the invention, a single-use robotic endoscope is provided. The robotic endoscope may be a gastroscope and can be the same as the steerable catheter assembly as described elsewhere herein. Traditional endoscopes can be complex in design and are usually designed to be re-used after procedures, which require thorough cleaning, dis-infection, or sterilization after each procedure. The existing endoscopes are often designed with complex structures to ensure the endoscopes can endure the cleaning, dis-infection, and sterilization processes. The provided robotic endoscope can be a single-use endoscope that may beneficially reduce cross-contamination between patients and infections. In some cases, the robotic gastroscope may be delivered to the medical practitioner in a pre-sterilized package and are intended to be disposed of after a single use.

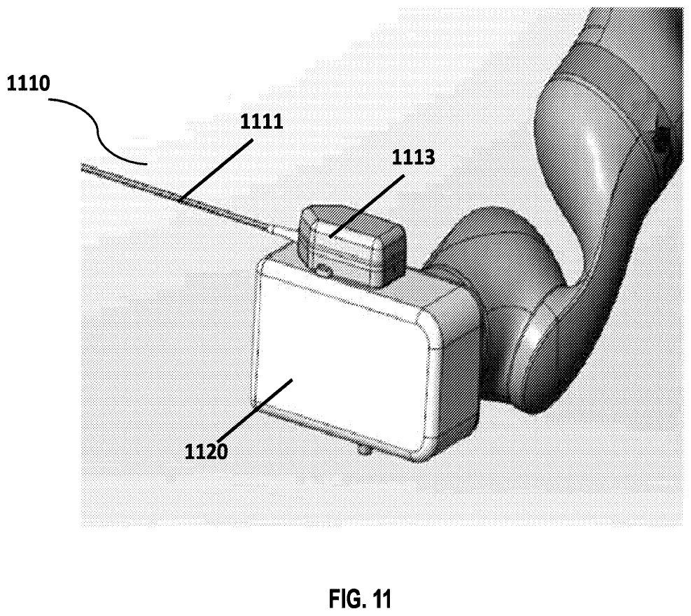

As shown in FIG. 11, a robotic endoscope (e.g., gastroscope or colonoscope) 1110 may comprise a handle portion 1113 and a flexible elongate member 1111. In some embodiments, the flexible elongate member 1111 may comprise a shaft, steerable tip, a steerable/active bending section and optionally an anti-prolapse passive section. The robotic gastroscope 1110 can be the same as the steerable catheter assembly as described in FIG. 10. The robotic gastroscope may be a single-use robotic endoscope. In some cases, only the catheter may be disposable. In some cases, at least a portion of the catheter may be disposable. In some cases, the entire robotic gastroscope may be released from the instrument driving mechanism and can be disposed of. In some cases, the gastroscope may contain varying levels of stiffness along its shaft, as to improve functional operation. In some cases, a minimum bend radius along the shaft may vary so that the kink resistance or anti-prolapse capability may be configurable along the length.

The robotic gastroscope can be releasably coupled to an instrument driving mechanism 1120. The instrument driving mechanism 1120 may be mounted to the arm of the robotic support system or to any actuated support system as described elsewhere herein. The instrument driving mechanism may provide mechanical and electrical interface to the robotic gastroscope 1110. The mechanical interface may allow the robotic gastroscope 1110 to be releasably coupled to the instrument driving mechanism. For instance, the handle portion of the robotic gastroscope can be attached to the instrument driving mechanism via quick install/release means, such as magnets and spring-loaded levels. In some cases, the robotic gastroscope may be coupled or released from the instrument driving mechanism manually without using a tool.

FIG. 12A and FIG. 12B shows an example of an instrument driving mechanism (IDM) 1220 providing a mechanical interface to the handle portion of the robotic endoscope. In some cases, the IDM 1220 for a robotic endoscope and one or more IDMs for one or more instruments (e.g., suturing instrument) 1231, 1233 may be attached to the robotic arm 1200. As shown in the example, the instrument driving mechanism (IDM) 1220 for the robotic endoscope may comprise a set of motors 1221 that are actuated to rotationally drive a set of pull wires of the flexible endoscope or catheter. The handle portion of the catheter assembly may be mounted onto the instrument drive mechanism 1220 so that its pulley assemblies or capstans are driven by the set of motors. The number of pulleys may vary based on the pull wire configurations. In some cases, one, two, three, four, or more pull wires may be utilized for articulating the flexible endoscope or catheter. Similarly, the instrument driving mechanism (IDM) 1231 for the suturing instrument herein may comprise a set of motors 1235 that are actuated to rotationally drive a set of pull wires of the suturing instrument thereby controlling the articulation of the bending sections of the suturing instrument, the roll movement and suture operation of the needle end effector as described above.

The handle portion may be designed allowing the robotic gastroscope to be disposable at reduced cost. For instance, classic manual and robotic gastroscopes may have a cable in the proximal end of the gastroscope handle. The cable often includes illumination fibers, camera video cable, and other optional sensor fibers or cables such as electromagnetic (EM) sensors, or shape sensing fibers. Such complex cable can be expensive, adding to the cost of the gastroscope. The provided robotic gastroscope may have an optimized design such that simplified structures and components can be employed while preserving the mechanical and electrical functionalities. In some cases, the handle portion of the robotic gastroscope may employ a cable-free design while providing a mechanical/electrical interface to the catheter.

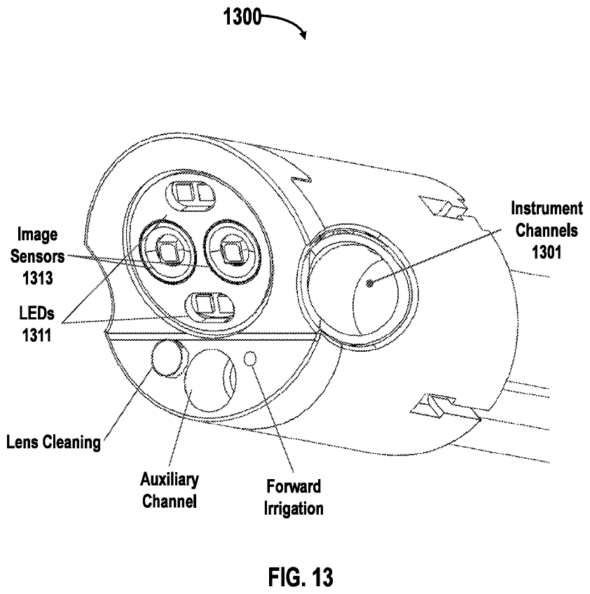

FIG. 13 shows an example of a distal tip 1300 of an endoscope. In some cases, the distal portion or tip of the endoscope 1300 may be substantially flexible such that it can be steered into one or more directions (e.g., pitch, yaw). The endoscope may comprise a tip portion, bending section, and insertion shaft. In some embodiments, the endoscope may have variable bending stiffness along the longitudinal axis direction. For instance, the endoscope may comprise multiple sections having different bending stiffness (e.g., flexible, semi-rigid, and rigid). The bending stiffness may be varied by selecting materials with different stiffness/rigidity, varying structures in different segments (e.g., cuts, patterns), adding additional supporting components or any combination of the above. In some embodiments, the endoscope may have variable minimum bend radius along the longitudinal axis direction. The selection of different minimum bend radius at different locations along the endoscope may beneficially provide anti-prolapse capability while still allowing the endoscope to reach hard-to-reach regions. In some cases, a proximal end of the endoscope needs not be bent to a high degree thus the proximal portion of the endoscope may be reinforced with additional mechanical structure (e.g., additional layers of materials) to achieve a greater bending stiffness. Such a design may provide support and stability to the endoscope. In some cases, the variable bending stiffness may be achieved by using different materials during extrusion of the endoscope. This may advantageously allow for different stiffness levels along the shaft of the endoscope in an extrusion manufacturing process without additional fastening or assembling of different materials.

The distal portion of the endoscope may be steered by one or more pull wires. The distal portion of the endoscope may be made of any suitable material such as co-polymers, polymers, metals or alloys such that it can be bent by the pull wires. In some embodiments, the proximal end or terminal end of one or more pull wires may be coupled to a driving mechanism (e.g., gears, pulleys, capstan etc.) via the anchoring mechanism as described above. The distal end or portion of one or more pull wires may be anchored or integrated to the distal portion of the endoscope, such that operation of the pull wires by the control unit may apply force or tension to the distal portion which may steer or articulate (e.g., up, down, pitch, yaw, or any direction in-between) at least the distal portion (e.g., flexible section) of the endoscope.

The endoscope may have a dimension so that one or more electronic components can be integrated to the endoscope. For example, the outer diameter of the distal tip may range from 3 mm to 25 mm, and the diameter of the instrument channels 1301 may range from 2 mm to 6 mm such that one or more instruments can be removably inserted through the endoscope to the surgical site. However, it should be noted that based on different applications, the outer diameter can be in any range smaller than 3 mm or greater than 25 mm, and the diameter of the instrument channels 1301 can be in any range such as about 4 mm or 5 mm to allow the suturing instrument herein passing through. The space not occupied by fluidics or instrument pass throughs can be used to embed electronic components into the wall of the endoscope.

The one or more electronic components may comprise an imaging device, illumination device or other optional sensors. In some embodiments, the imaging device may be a video camera 1313. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors.

The illumination device may comprise one or more light sources 1311 positioned at the distal tip. The light source may be a light-emitting diode (LED), an organic LED (OLED), a quantum dot, or any other suitable light source. In some cases, the light source may be a miniaturized LED for a compact design or Dual Tone Flash LED Lighting.

The imaging device and the illumination device may be integrated to the endoscope. For example, the distal portion of the endoscope may comprise suitable structures matching at least a dimension of the imaging device and the illumination device. The imaging device and the illumination device may be embedded into the catheter. A camera may be located at the distal portion 1300. The distal tip may have a structure to receive the camera, and illumination device. For example, the camera may be embedded into a cavity at the distal tip of the catheter. The cavity 1410 may be integrally formed with the distal portion of the cavity and may have a dimension matching a length/width of the camera such that the camera may not move relative to the endoscope. The camera may be adjacent to one or more instrument channels 1301 of the endoscope to provide near field view of the tissue or the organs. In some cases, the attitude or orientation of the imaging device may be controlled by controlling a rotational movement (e.g., roll) of the endoscope.

The power to the camera may be provided by a wired cable. In some cases, the cable wire may be in a wire bundle providing power to the camera as well as illumination elements or other circuitry at the distal tip of the endoscope. The camera and/or light source may be supplied with power from a power source located at the handle portion via wires, copper wires, or via any other suitable means running through the length of the catheter. In some cases, real-time images or video of the tissue or organ may be transmitted to an external user interface or display wirelessly. The wireless communication may be WiFi, Bluetooth, RF communication or other forms of communication. In some cases, images or videos captured by the camera may be broadcasted to a plurality of devices or systems. In some cases, image and/or video data from the camera may be transmitted down the length of the catheter to the processors situated in the handle portion via wires, copper wires, or via any other suitable means. The image or video data may be transmitted via the wireless communication component in the handle portion to an external device/system. In some cases, the system may be designed such that no wires are visible or exposed to operators.

In conventional endoscopy, illumination light may be provided by fiber cables that transfer the light of a light source located at the proximal end of the endoscope, to the distal end of the robotic endoscope. In some embodiments of the disclosure, miniaturized LED lights may be employed and embedded into the distal portion of the catheter to reduce the design complexity. In some cases, the distal portion may comprise a structure having a dimension matching a dimension of the miniaturized LED light source. As shown in the illustrated example, two cavities may be integrally formed with the endoscope to receive two LED light sources 1311. For instance, the outer diameter of the distal tip may range from 3 mm to 25 mm and diameter of the working channel of the endoscope may be around 4.5 or 6 mm such that two LED light sources may be embedded at the distal end. The outer diameter can be in any range smaller than 3 mm or greater than 25 mm, and the diameter of the instrument channels 1301 can be in any range according to the tool's dimensional or specific application. Any number of light sources may be included. The internal structure of the distal portion may be designed to fit any number of light sources.

In some cases, each of the LEDs may be connected to power wires which may run to the proximal handle. In some embodiments, the LEDs may be soldered to separated power wires that later bundle together to form a single strand. In some embodiments, the LEDs may be soldered to pull wires that supply power. In other embodiments, the LEDs may be crimped or connected directly to a single pair of power wires. In some cases, a protection layer such as a thin layer of biocompatible glue may be applied to the front surface of the LEDs to provide protection while allowing light emitted out. In some cases, an additional cover may be placed at the forwarding end face of the distal tip providing precise positioning of the LEDs as well as sufficient room for the glue. The cover may be composed of transparent material matching the refractive index of the glue so that the illumination light may not be obstructed.

The working channel (e.g., instrument channel 1301, auxiliary channel) may be designed to provide protection for the internal components such as flexible instruments (e.g., suturing instrument, forceps, etc.). When flexible instruments pass through a conventional working channel, they may be obstructed by the working channel due to kinking, ovalizing and/or high friction force. The working channel may provide a high hoop strength and a capability of achieving low bend radius. The working channel may also be designed to provide low friction in the inner surface. The suturing instrument as described herein may be passed through the working channel and advanced over the distal tip of the endoscope or retracted back into the working channel.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A suturing instrument comprising:
a flexible shaft comprising an articulatable bending section; and
a needle driver located at a distal end of the bending section, wherein the needle driver comprises a first jaw driven to have a translatable movement relative to a second jaw thereby rolling a needle grasped by the first jaw and the second jaw into a desired orientation.

2. The suturing instrument of claim 1, wherein the first draw is driven to translate relative to the second draw by a first pull wire that is extended through the flexible shaft.

3. The suturing instrument of claim 2, wherein a proximal end of the first pull wire is coupled to a handle of the suturing instrument.

4. The suturing instrument of claim 3, wherein the handle is releasably coupled to a robotic support via a first instrument driving mechanism.

5. The suturing instrument of claim 2, wherein the first draw or the second draw is driven to open or close by a second pull wire that is extended through the flexible shaft.

6. The suturing instrument of claim 1, wherein the suturing instrument is inserted through a working channel of a flexible robotic endoscope.

7. The suturing instrument of claim 6, wherein the flexible robotic endoscope comprises an articulable bending section.

8. The suturing instrument of claim 7, wherein the flexible robotic endoscope is releasably coupled to a robotic support via a second instrument driving mechanism.

9. The suturing instrument of claim 8, wherein the articulable bending section is actuated by the second instrument driving mechanism.

10. The suturing instrument of claim 1, wherein the needle is rolled into the desired orientation by an angle.

11. The suturing instrument of claim 10, wherein a range of the angle is determined based on a translational travel distance between the first draw and the second draw.

12. A robotic endoluminal suturing instrument comprising:
a flexible shaft comprising an articulatable bending section, wherein an articulating motion of the articulatable bending section is driven by a first pull wire;
a needle driver located at a distal end of the bending section, wherein the needle driver comprises a first jaw driven by a second pull wire to have a translatable movement relative to a second jaw thereby rolling a needle grasped by the first jaw and the second jaw into a desired orientation; and
a handle configured to releasably couple the robotic endoluminal suturing instrument to an instrument driving mechanism, wherein the instrument driving mechanism actuates the first pull wire and the second pull wire.

13. The robotic endoluminal suturing instrument of claim 12, wherein the handle is releasably coupled to a robotic support via a first instrument driving mechanism.

14. The robotic endoluminal suturing instrument of claim 12, wherein the first draw or the second draw is driven to open or close by a third pull wire that is extended through the flexible shaft.

15. The robotic endoluminal suturing instrument of claim 12, wherein the robotic endoluminal suturing instrument is inserted through a working channel of a flexible robotic endoscope.

16. The robotic endoluminal suturing instrument of claim 15, wherein the flexible robotic endoscope comprises an articulable bending section.

17. The robotic endoluminal suturing instrument of claim 16, wherein the flexible robotic endoscope is releasably coupled to a robotic support via a second instrument driving mechanism.

18. The robotic endoluminal suturing instrument of claim 17, wherein the articulable bending section is actuated by the second instrument driving mechanism.

19. The robotic endoluminal suturing instrument of claim 12, wherein the needle is rolled into the desired orientation by an angle.

20. The robotic endoluminal suturing instrument of claim 19, wherein a range of the angle is determined based on a translational travel distance between the first draw and the second draw.
